(19) United States
(12) Patent Application Publication
(10) Pub. No.: US 2018/0161546 A1
(43) Pub. Date: Jun. 14, 2018

(54) INTRAVENOUS CATHETER STOPPER

(71) Applicants: National Guard Health Affairs, Riyadh (SA); King Saud bin Abdulaziz University for Health Sciences, Riyadh (SA); King Abdullah International Medical Research Center, Riyadh (SA)

(72) Inventors: Muhammad Ovais ASLAM, Madinah (SA); Muhammad Arqam AWAIS, Lahore (PK); Minahil AWAIS, Lahore (PK)

(73) Assignees: National Guard Health Affairs, Riyadh (SA); King Saud bin Abdulaziz University for Health Sciences, Riyadh (SA); King Abdullah International Medical Research Center, Riyadh (SA)

(21) Appl. No.: 15/376,029

(22) Filed: Dec. 12, 2016

(51) Int. Cl. A61M 25/06; A61M 39/20; A61M 39/10

(52) U.S. Cl. CPC A61M 25/0606; A61M 39/10; A61M 39/20

(57) ABSTRACT

A stopper is provided for preventing blockage of a cannula of an intravenous catheter in-between uses including a hub having a connector for securing to the catheter, and a stylet having a length and a shape configured to fill a lumen of the cannula, where the hub is configured to place the stylet within the lumen of the cannula, and where the stylet is configured to prevent blood from clotting within the lumen of the cannula when inserted in a patient between uses. In some implementations, the stopper can have a movable stylet. In an example, the stylet can have a variable stiffness configured to aid in moving and prevent blockage of the lumen by kinking of the cannula.

### BACKGROUND

Intravenous catheters are used for intravenous (IV) access and providing IV therapy such as injection of medicine and/or fluid to a patient. IV therapy is beneficial for several conditions and medical situations including dehydration, nutrition, physiological shock, surgery, blood transfusions, chemotherapy and medication administration. There are many types of IV catheters that can be used for peripheral and/or central IV use. An example of an IV catheter is an over-the-needle catheter having a cannula that is inserted into a vein via a needle/trocar. The needle/trocar is inserted through a lumen of the cannula, where the needle/trocar is inserted into the vein and then withdrawn, leaving the cannula within the vein. IV catheters can be used intermittently; however, difficulty can arise when the cannula is blocked.

### SUMMARY

A stopper is provided for preventing blockage of a cannula of an intravenous catheter in-between uses including a hub having a connector for securing to the catheter, and a stylet having a length and a shape configured to fill a lumen of the cannula, where the hub is configured to place the stylet within the lumen of the cannula, and where the stylet is configured to prevent blood from clotting within the lumen of the cannula when inserted in a patient between uses. In some implementations, the stopper can have a movable stylet. In an example, the stylet can have a variable stiffness configured to aid in moving and prevent blockage of the lumen by kinking of the cannula.

### BRIEF DESCRIPTION OF THE DRAWINGS

A more complete appreciation of the disclosed embodiments and many of the attendant advantages thereof will be readily obtained as the same becomes better understood by reference to the following detailed description when considered in connection with the accompanying drawings, wherein:

FIG. 1A is a drawing of a cross-section of a stopper having a stylet coupled to an intravenous catheter having a cannula, where the stylet is extended through a lumen of the cannula according to an example;

FIG. 1B is a drawing of a cross-section of a stopper having a retractable stylet and the intravenous catheter of FIG. 1A where the retractable stylet is withdrawn from the lumen according to an example;

FIG. 1C shows a hand of a patient having the cannula of the IV catheter inserted in a vein and the stylet of the stopper within the lumen of the cannula according to an example;

FIG. 2A is a drawing of a cross-section of an intravenous catheter having a cannula, a catheter connector, and a pair of tabs according to an example;

FIG. 2B a drawing of a cross-section of an intravenous catheter having a cannula, a catheter connector including a valve, and a pair of tabs according to an example;

FIG. 3A is a drawing of a cross-section of a stopper having a fixed stylet according to an example;

FIG. 3B is a drawing of a cross-section of a stopper having a rubber membrane, a retractable stylet, and a stopper connector, where the retractable stylet is extended past the stopper connector according to an example;

FIG. 3C is a drawing of a cross-section of the stopper shown in FIG. 3B where the retractable stylet is withdrawn from extending beyond the stopper connector according to an example;

FIGS. 3D-3E are drawings of a cross-section of a stopper having a roller, a retractable stylet, and a stopper connector according to an example;

FIGS. 3F-3G are drawings of a cross-section of a stopper having a coiling retractor, a coiling stylet, and a stopper connector according to an example;

FIG. 4A is a drawing of a stopper having a protective cap for covering the stylet when extended and not in use according to an example;

FIG. 4B is a drawing of a stopper having a protective cap for covering the stylet when retracted and not in use according to an example;

FIG. 4C is a drawing of the coiling retractor of the stopper in FIGS. 3F-3G including an anti-coagulant agent according to an example;

FIGS. 5A-B show pictures of the stopper used with an IV catheter having a two-way connector according to an example;

FIG. 6A shows a cross-section along a width of a stylet within a lumen of a catheter according to an example;

FIG. 6B shows a cross-section along a width of a stylet and a catheter having tongue and groove complementary features according to an example;

FIG. 6C shows a cross-section along a length of a stylet and a catheter having head and socket complementary features according to an example;

FIG. 7 shows a picture demonstrating how an injection by a syringe can be administered to the cannula by removing the stylet of the stopper according to an example; and

FIG. 8 shows a table listing ranges of sizes and calibers of the cannula in gauge according to an example.

### DETAILED DESCRIPTION

The present disclosure preferably can be used for IV catheters having a cannula for peripheral applications with intermittent use or infusion. In some cases, intermittent infusion is used when a patient requires IV therapy such as medication only at certain times, and does not require additional fluid.

IV catheters can be used with other IV therapy devices and equipment configured for intermittent use such as a syringe or tubing for an intravenous drip (e.g., pump or gravity drip). After the IV therapy is complete, the tubing is disconnected from the IV catheter and a protective cap is applied to the IV catheter. Difficulty can arise when the cannula is again fixed with the syringe or tubing.

In between uses, blood can clot within the lumen of the cannula so there is a need to “flush” the cannula before administering subsequent IV therapy. In some cases, when the clotted blood within the cannula is cleared with the flush, the clotted blood travels into the blood stream of the patient and can be lodged into the patient's lungs and lead to serious complications such as a pulmonary embolism. In some instances, the clotted blood can travel to the brain and lead to an infarction leading to weakness of a part of the body. In other cases it's not possible to dislodge the clot from the lumen of the cannula so the cannula has to be replaced. Replacing the cannula is painful, requires another peripheral vein location, a trained medical person to replace the catheter, and adds cost for another cannula.

Another common problem with IV cannulas is kinking. When the cannula is inserted into a vein in a part of the body that bends, such as the elbow or wrist, then a splint may be required to prevent the cannula from bending. Once the cannula is kinked, the cannula becomes blocked and the IV therapy cannot be administered. Similar to clotted cannulas, blocked cannulas need to be replaced.

Referring now to the drawings, wherein like reference numerals designate identical or corresponding parts throughout the several views.

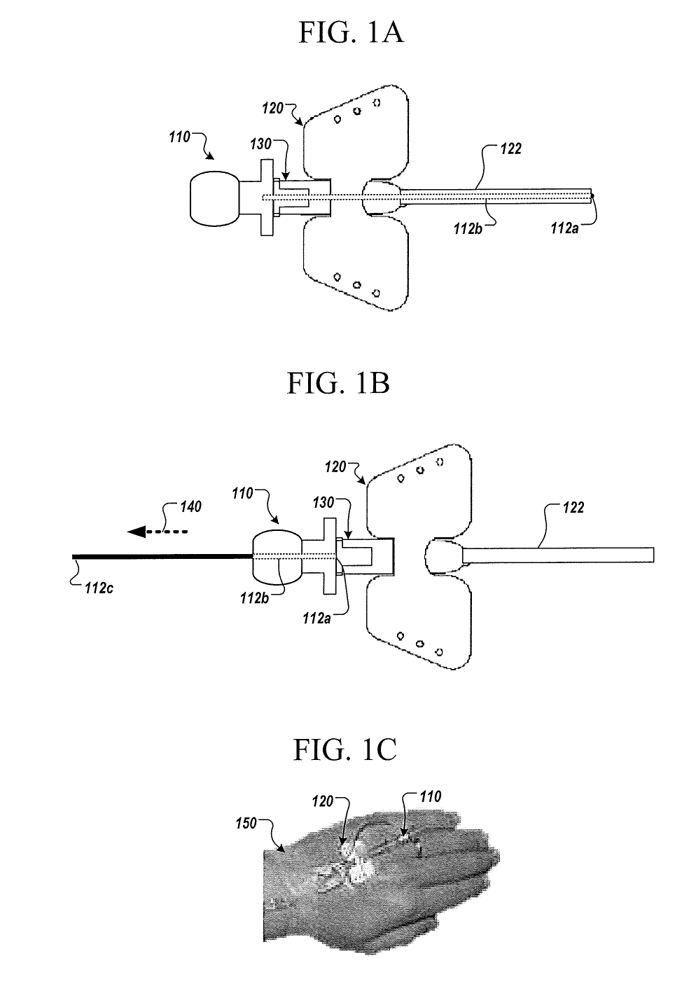

FIG. 1A is a drawing of a cross-section of a stopper 110 having a stylet 112 coupled to an intravenous (IV) catheter 120 having a cannula 122, where the stylet 112 is extended through a lumen of the cannula 122 according to an example. The extension of a distal end 112a of the stylet 112, which is preferably rounded, is configured to at least substantially be equal to a distal end of the cannula 122 and is configured to prevent blockage of the lumen between uses of the IV catheter 120 in several ways.

In an example, the stylet 112 is coated with an anti-clotting agent configured to prevent blockage of the lumen. The stopper 110 and the stylet 112 can be made from similar biocompatible materials used in the IV catheter 120 such as polyurethane and PVC according to an example. In an example, the stylet 112 is configured to prevent blockage of the lumen by creating a physical obstruction of the lumen. In an example, the stylet 112 has a stiffness configured to strengthen the cannula 122 and to prevent blockage of the lumen by kinking of the cannula 122.

In some implementations, the stylet 112 can have a varying stiffness along a stylet length. The stylet 112 can be configured to have a variable stiffness using a single material and different mechanical properties such as an aeration density and a varying physical structure. Alternatively, the stylet 112 can be configured to have a variable stiffness using a combination of materials with different hardness.

In an example, the stylet 112 can have a stiff proximal portion to aid in movement and prevent kinking and a soft or flexible distal portion within the cannula 122. In an example, the varying stiffness along the length of the stylet 112 can be alternating segments of increased compliance and increased rigidity (not shown). In an example, the stiffness of the stylet 112 is complementary to the cannula 122 such that a combined stiffness of the stylet 112 and the cannula 122 is configured to prevent patient discomfort. In an example, a distal portion of the stylet 112 can have a lesser stiffness than a proximal portion of the stylet to prevent kinking of the cannula 122 and injury to tissue when the stylet 112 is inserted in the cannula 122. In an example, a proximal portion of the stylet 112 can have a greater stiffness than a distal portion of the stylet 112 to aid in extending and retracting the stylet 112.

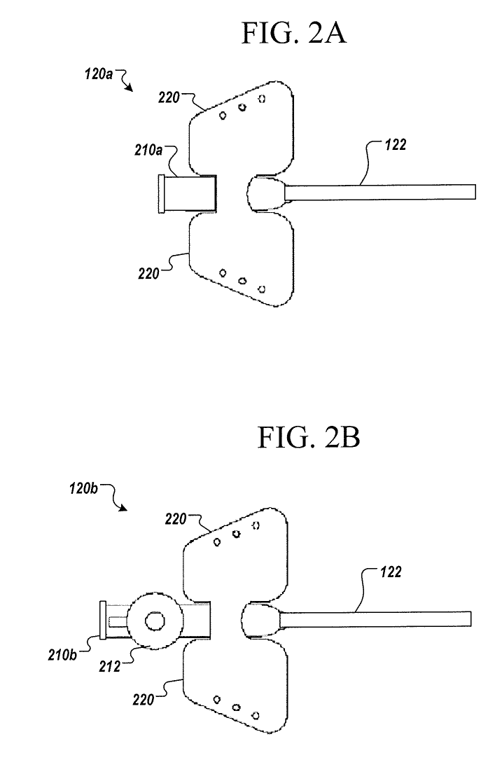

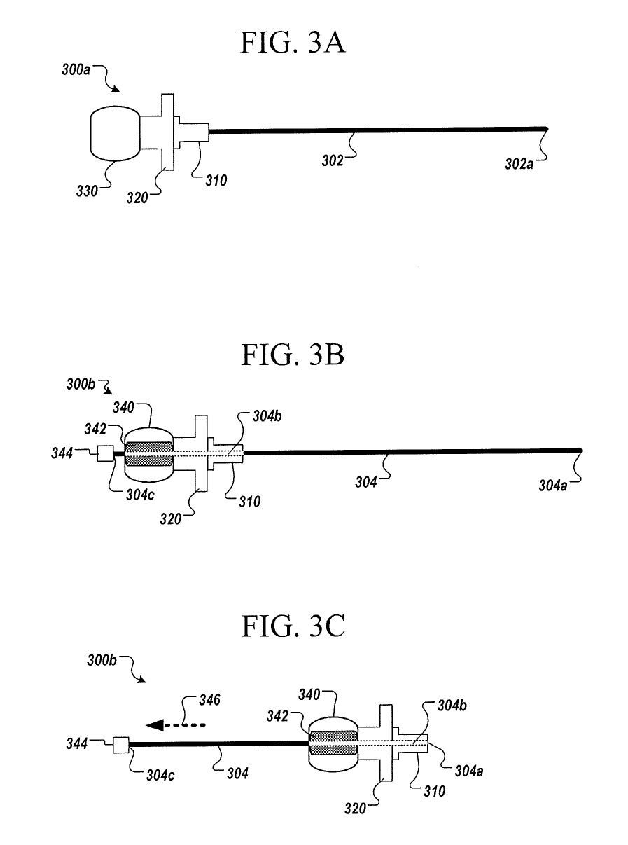

In an example, a connecting portion of the stopper 110 (See 310 in FIG. 3A-3G) can have a complementary shape to a connecting portion of the IV catheter 120 (See 210a-b in FIG. 2A-2B) configured to form a flashback chamber 130. The flashback chamber 130 is preferably transparent such that a proximal end of the cannula 122 can be visible.

FIG. 1B shows a drawing of a cross-section of the stopper 110 connected to the IV catheter 120, where the stylet 112 is withdrawn from the cannula 122 in a direction 140. In some implementations, the stopper 110 can have a retractable stylet 300c-d configured to enter and to withdraw from the lumen of the cannula 122 of the IV catheter 120. (See FIGS. 3B-3G)

FIG. 1C shows a hand 150 of a patient with the IV catheter 120 and the stopper 110 according to an example. The stylet 112 of the stopper 110 is within the lumen of the cannula which is inserted in a vein of the patient.

### IV Catheter

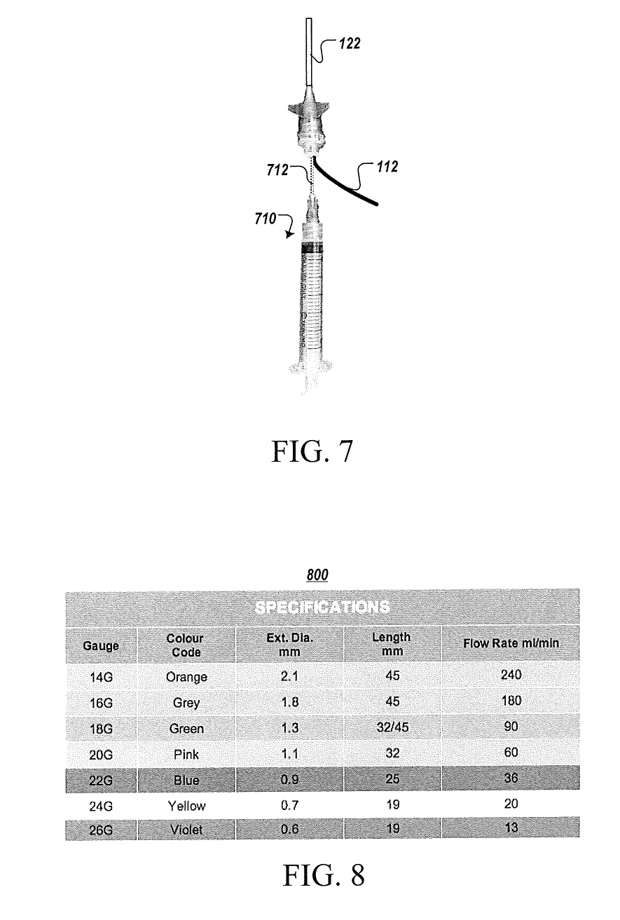

FIG. 2A is a drawing of a cross-section of an IV catheter 120a having a cannula 122 configured to be inserted into the vein, a catheter connector 210a configured to interface with IV therapy devices and equipment (e.g., the stopper 110), and a pair of tabs 220 for securing the IV catheter 120a to a patient's skin according to an example. FIG. 2B is drawing of a cross-section of an IV catheter 120b having a cannula 122, a catheter connector 210b that includes a flow control valve 212, and a pair of tabs 220 according to an example. The cannula 122 can have different lengths and lumen sizes which can be described by a gauge caliber. FIG. 8 shows a table 800 listing sizes and calibers of the cannula 122 in gauge ranging from size 26 gauge (smallest) to size 14 gauge (largest) according to an example. In an example, the stopper 110 can be used with any commercially available I/V catheter having a cannula and can be provided in the market as a disposable with a color coding scheme according to a size of the lumen of the cannula and/or a flow rate.

### Stopper

In some implementations, a shape and length of the stylet 112 is configured to prevent crystallization of medicine and blood from clotting within the lumen of the cannula 122 when inserted in a patient between uses of the IV catheter 120. The lengths of small gauge cannulas are kept small to prevent clotting of blood and blockage of the cannula 122. Using the stopper 110, new cannulas having longer lengths can be manufactured and used for patient care.

The stylet 112, when inserted into the lumen of the cannula 122, is configured to prevent an incidence of blood clotting within the lumen of the cannula 122. In an example, the stylet 112 is configured to have a shape and dimension to fill the lumen of the cannula 122, as well as a length equal to the cannula 122.

FIGS. 3A-3F are drawings of cross-sections of different examples of the stopper 110 and are represented as 300a-300d, respectively. Each stopper 300a-d can include a stopper connector 310 configured to securely mate with the catheter connector 210 of the IV catheter 120 and a lip 320 for aid in connecting the stopper 110, 300a-d to the IV catheter 120.

### Fixed Stylet

FIG. 3A is a drawing of a cross-section of a stopper 300a having a fixed stylet 302 that is fixed to either the stopper connector 310 or a hub 330 according to an example. As shown in FIG. 1A, when the stopper 300a is inserted into the IV catheter 120, the distal end (302a) of the stylet 302 is configured to be flush with the distal end of the cannula 122.

### Rubber Membrane

FIGS. 3B-3C are drawings of a cross-section of a stopper 300b having a hub 340 with a rubber membrane 342, a sliding stylet 304, and a stopper connector 310, where the sliding stylet 304 is movable according to an example. A distal end 304a of the sliding stylet 304 of the stopper 300b can be extended past the stopper connector 310 (FIG. 3B) or retracted 346 behind the stopper connector 310 (FIG. 3C). In an aspect, the rubber membrane 342 is configured to grip the sliding stylet 304 and prevent the sliding stylet 304 from moving freely. Optionally, a proximal end 304c of the sliding stylet 304 can include a handle 344. The handle 344 can be used to by the user to manually pulling and pushing the sliding stylet 304.

### Roller

FIGS. 3D-3E are drawings of a cross-section of a stopper 300c having a hub 350 with a roller 352, a cogged stylet 306, and the stopper connector 310, where the roller 352 is configured to extend or retract a cogged stylet 306 by creating traction according to an example. In an example, a surface of the cogged stylet 306 can have a plurality of cogs 307 configured to aid in creating traction. In an example, the roller 352 includes a plurality of teeth 353 that are complementary to the plurality of cogs 307 of the cogged stylet 306. In an example, a rotation of the roller 352 in a first direction 354 results in an extension 355 of the cogged stylet 306, and a rotation in an opposite direction 356 results in a retraction 357. When the roller 352 is rotated, the cogged stylet 306 is moved distally or proximally from the lumen of the cannula 122. The roller 352 can be rotated by a user's thumb or finger. In an example, the roller 352 can be configured to turn a gear (not shown) with a set of complementary teeth with respect to the plurality of cogs of the cogged stylet 306.

### Coiling Retractor

FIGS. 3F-3G are drawings of a cross-section of a stopper 300d having a hub 360 with a coiling retractor 362, a coiling stylet 308, and the stopper connector 310, where the coiling retractor 362 is configured to coil the coiling stylet 308 within the coiling retractor 362, according to an example. In an example, a proximal end 308c of the coiling stylet 308 can be attached to a portion of the coiling retractor 362 such that a rotation of the coiling retractor 362 in a first direction 364 results in an extension 365 of the coiling stylet 308, and a rotation in an opposite direction 366 results in a retraction 367. When the coiling retractor 362 is rotated, the coiling stylet 308 is moved distally or proximally from the lumen of the cannula 122. In an example, the coiling retractor 362 can include a spring configured to automatically recoil or to extend the coiling stylet 308. In some implementations, the coiling retractor 362 can be filled partially or fully with an anti-coagulant agent to coat the coiling stylet 308, which can further prevent clotting.

### Protective Cap

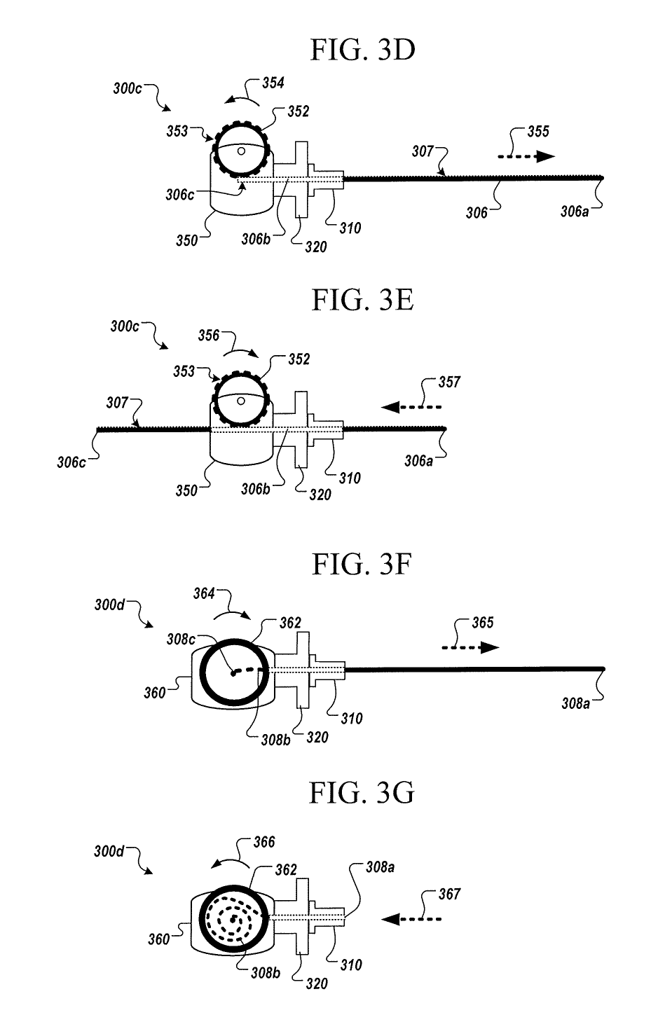

FIG. 4A is a drawing of a stopper 110 having a protective cap 410 for covering the stylet 112 when extended and not in use according to an example. FIG. 4B is a drawing of a stopper 110 having a protective cap 420 for covering the stylet 112 when retracted and not in use according to an example. In some implementations, the protective cap 410, 420 can be filled partially or fully with an anti-coagulant agent 402 configured to coat the stylet 112 and further prevent clotting of the lumen. In an example, the stopper 300a can connect to a protective cap 410 at the stopper connector 310 in order to cover the fixed stylet 302 when not in use according to an example (See FIG. 4A). In an example, the stopper 300b-c can have a protective cap 420 connected at the hub 340, 350 with or without the anti-coagulant agent 402 for covering and coating respectfully, the sliding stylet 304 or the ribbed stylet 306 when not in use according to an example (See FIG. 4B). In an example, the hub 360 of the coiling retractor 362 can serve as a protective cap 430 and include the anti-coagulant agent 402 for coating the coiling stylet 308 when the coiling stylet 308 is retracted according to an example (See FIG. 4C).

### Two-Way Connector

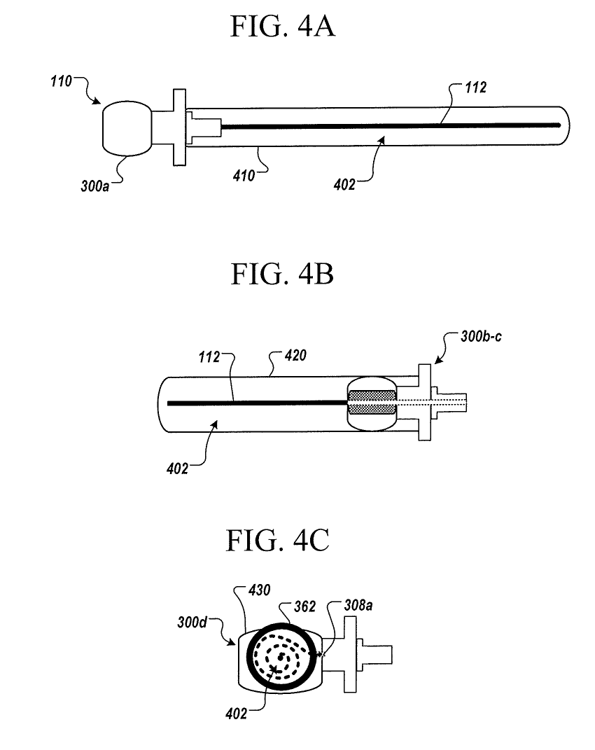

FIGS. 5A-B show pictures of the stopper 110 used with an IV catheter 510 having a two-way connector according to some implementations. In an example, the IV catheter 510 can have a first connector 512 and a second connector 514. In an example, the IV catheter 510 can extend (FIG. 5A) and retract (FIG. 5B) the stylet 112 from a lumen of the first connector 512, while an IV therapy is administered from the second connector 514. The second connector could also be polluted with the anti-coagulant agent 402 onto the stylet 112 as it is being extended or retracted. Accordingly, the location of the second connector 514 provides distinct advantages of being able to directly provide IV therapy at a point of entry.

### Stylet and Catheter Complementary Features

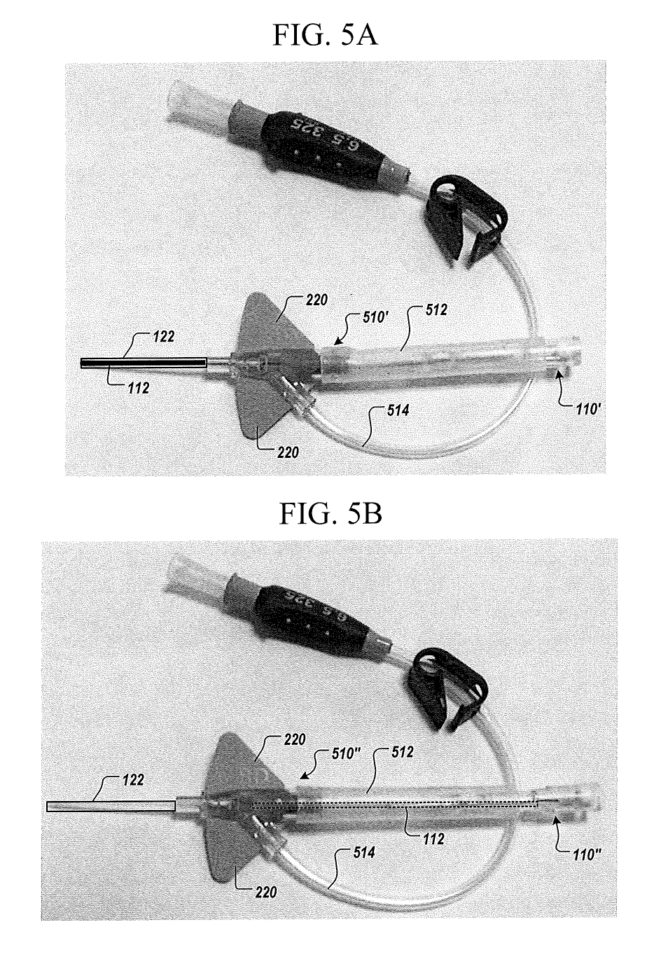

FIG. 6A shows a cross-section along a width of a stylet 620a within a lumen 612 of a cannula 610a according to an example. The stylet 620a is shown having complementary cylindrical shape with the lumen 612 of the cannula 610a. In some implementations, the stylet 112 and/or the cannula 122 can have complementary features configured to prevent the stylet 112 from being entirely removed from the cannula 122 when the stylet 112 is retracted, as well as to facilitate alignment the stylet 112 with the lumen of the cannula 122 when the stylet 112 is fully retracted from the cannula 122.

FIG. 6B shows a cross-section along a width of a stylet 620b and a cannula 610b which form a tongue and groove complementary feature 630 according to an example. The tongue and groove complementary feature 630 can allow for alignment of the stylet 620b into the cannula 610b by aligning a groove 622 in the stylet 620b with a tongue 614 of the cannula 610b. In an example, the groove 622 can have a lip 624 and the tongue 614 can include a head 616 configured to catch and to guide the stylet 620b. In some implementations, the complementary feature 630 can be configured with the stylet 620b within the stopper 110. In some implementations, the stylet 620b can be configured with the groove and the cannula 610b can be configured with the tongue. FIG. 6C shows a cross-section along a length of a stylet 620c and a cannula 610c which form a head and socket complementary feature 640 according to an example. The head and socket complementary feature 640 can include a flush stylet head 626 configured to be caught in an indented wall 618 of a cannula 610c. In an example, the flush stylet head 626 of the distal portion of the stylet 620c is wider than the indented wall 618 of the cannula 610c such that the stylet 620c is prevented from being fully removed from the cannula 610c while also allowing the lumen of the cannula 610c to be unobstructed.

### Advantages

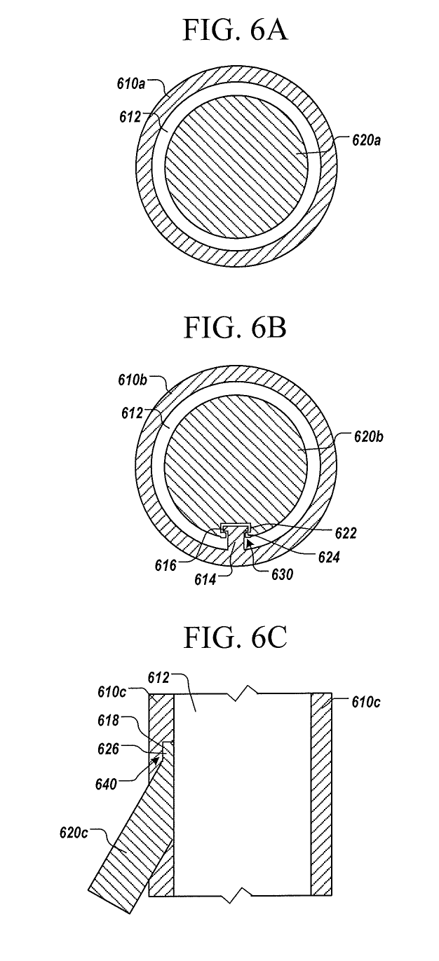

As shown in FIG. 7, the stylet 112 can be retracted from the lumen of the cannula 122 and a needle 712 of a syringe 710 can be introduced into the lumen of the cannula 122 in order to administer the IV therapy. In an example, the flashback chamber 130 formed by connecting the stopper 110 and the catheter 120 can have a width to house both the stylet 112 and the needle 712. In an example, the flashback chamber 130 can be transparent to aid the user in aligning the needle 712 to the lumen of the cannula 122. Importantly, the IV therapy can be administered without disconnection of the stopper 110 from the catheter 120.

Obviously, numerous modifications and variations of the present disclosure are possible in light of the above teachings. It is therefore to be understood that within the scope of the appended claims, the invention may be practiced otherwise than as specifically described herein.

1. A stopper for an intravenous catheter, the stopper comprising:
a hub having an interior channel and being configured to secure the stopper to the catheter;
a connector having an interior channel, the connector being connected to the hub;
a stylet located within the interior channel of the hub and the connector and having a length and shape configured to fill a lumen of a cannula of the intravenous catheter, wherein the stylet is configured to prevent fluid from clotting within the catheter.

2. The stopper of claim 1, wherein the stylet includes a distal portion which is substantially equal to a distal end of the cannula when the hub is secured to a complementary connector of the catheter.

3. The stopper of claim 1, wherein the hub includes a rubber membrane configured to grip the stylet within the interior channel of the hub.

4. The stopper of claim 1, wherein
the stylet includes a plurality of cogs along a length of the stylet,
the hub includes a roller configured to engage with the plurality of cogs of the stylet, and
the roller is configured to extend or retract the stylet within the interior channels and the lumen of the cannula in response to a rotation of the roller and engagement with the plurality of cogs.

5. The stopper of claim 1, wherein the stylet has a length configured for a catheter having a two-way connector.

6. The stopper of claim 1, wherein the stylet has a variable stiffness along a length of the stylet.

7. The stopper of claim 6, wherein a distal portion of the stylet has a lesser stiffness than a proximal portion of the stylet to prevent kinking of the cannula and injury to tissue when the stylet is inserted in the cannula.

8. The stopper of claim 6, wherein a proximal portion of the stylet has a greater stiffness than a distal portion of the stylet to aid in extending and retracting the stylet.

9. The stopper of claim 1, wherein the hub includes a coiling retractor configured to coil the stylet within the hub.

10. The stopper of claim 9, wherein the coiling retractor is configured to extend the stylet from the coiling retractor or retract the stylet into the coiling retractor in response to rotation of the coiling retractor.

11. The stopper of claim 9, wherein the coiling retractor includes an anti-coagulant agent configured to coat the stylet.

12. The stopper of claim 1, further comprising:
a protective cap configured to enclose the stylet.

13. The stopper of claim 12, wherein the protective cap includes an anti-coagulant agent configured to coat the stylet.

14. The stopper of claim 12, wherein the protective cap is connected to the connector and configured to enclose a distal end of the stylet when the stylet is extended from a distal end of the stopper.

15. The stopper of claim 12, wherein the protective cap is connected to the hub and configured to enclose a proximal end of the stylet when the stylet is retracted from a distal end of the stopper.

16. The stopper of claim 1, wherein the stylet includes at least one groove and the cannula includes at least one tongue projection, wherein, when the stylet is inserted into the lumen of the cannula, a groove of the stylet is configured to align with a respective tongue projection of the cannula.

17. The stopper of claim 1, wherein the interior channel of the hub and the interior channel of the connector are aligned with each other in a direction of insertion of the stylet.

18. An intravenous catheter and stopper assembly, the assembly comprising:
an intravenous catheter including
a cannula having a lumen,
a catheter connector having a first end connected to the cannula and a second end configured to allow access to the lumen, and
a pair of tabs for securing the catheter to a patient's skin; and
a stopper including
a hub having an interior channel and being configured to secure the stopper to the catheter,
a stopper connector having a complementary shape to the catheter connector and an interior channel, the stopper connector being connected to the hub,
a stylet located within the interior channel of the hub and the stopper connector and having a length and shape configured to fill the lumen of the cannula, wherein
the stylet connector is configured to secure to the catheter connector, and
the stylet is configured to prevent fluid from clotting within the catheter.
